(19) United States
(12) Patent Application Publication
(10) Pub. No.: US 2009/0234245 A1
(43) Pub. Date: Sep. 17, 2009

(54) NON-INVASIVE MONITORING OF INTRACRANIAL PRESSURE

(75) Inventors: Richard A. JAFFE, Palo Alto, CA (US); Jaime R. Lopez, El Granada, CA (US); Xuefeng Cheng, Cupertino, CA (US); Paul Chan, Sunnyvale, CA (US)

Correspondence Address: COOPER & DUNHAM, LLP, 30 Rockefeller Plaza, 20th Floor, NEW YORK, NY, 10112, US

(73) Assignee: O2 MedTech, Inc., Los Altos, CA (US)

(21) Appl. No.: 12/049,843

(22) Filed: Mar. 17, 2008

(51) Int. Cl. A61B 5/00

(52) U.S. Cl. 600/561

(57) ABSTRACT

Methods, systems, and related computer program products for are described for non-invasive detection of intracranial pressure (ICP) variations in an intracranial compartment of a patient. Optical radiation is propagated transcranially into the intracranial compartment, and optical radiation that has migrated through at least a portion of the intracranial compartment and back out of the cranium is detected. At least one signal representative of the detected optical radiation is processed to extract therefrom at least one component signal that varies in time according to at least one of an intrinsic physiological oscillation and an externally driven oscillation in the patient. Examples of suitable intrinsic physiological oscillations include intrinsic respiratory and cardiac oscillations. Examples of suitable externally driven oscillations include ventilated respiratory oscillations and externally mechanically induced oscillations. The extracted component signal is then processed to generate an output signal representative of the ICP variations in the intracranial compartment.

### FIELD

This patent specification relates to the monitoring of a physiological condition of a patient using non-invasive measurement techniques. More particularly, this patent specification relates to the monitoring of intracranial pressure (ICP) using non-invasive optical techniques.

### BACKGROUND AND SUMMARY OF THE DISCLOSURE

Intracranial pressure refers to the pressure exerted by the cranium on the tissue and fluid matter contained inside the cranium, which includes brain tissue, cerebrospinal fluid, and blood circulating in the brain. Typical values of ICP for a patient at rest are in the range of 10-15 mm Hg (0.013-0.020 atm). Elevated ICP levels are generally undesirable and are often a result of a traumatic head injury, an infectious disease such as meningitis, or another pathological condition such as brain tumor. For an adult, an elevated ICP above 40 mm Hg is likely to cause severe harm, and even pressures between 25 and 30 mm Hg are usually fatal if prolonged. Detection of ICP variations is recognized as an important tool in monitoring the state of injured patients, diagnosing symptoms of potentially diseased patients, and monitoring patient health during surgery or other therapeutic interventions.

Although various proposals have been made for non-invasive ICP monitoring, it is still generally recognized that reliable detection of ICP variations requires invasive measurement devices. However, such invasive techniques involve exposing and potentially traumatizing the brain tissue, which can increase the risk of infection, hemorrhage, leakage of cerebrospinal fluid, and other problems that can actually worsen the patient's condition.

Described in this patent specification are methods, systems, and related computer program products for non-invasive detection of ICP variations using optical techniques in the visible and/or near infrared regime. According to one preferred embodiment, optical radiation is propagated transcranially into the intracranial compartment, and optical radiation is detected that has migrated through at least a portion of the intracranial compartment and back out of the cranium. At least one signal representative of the detected optical radiation is processed to extract therefrom at least one component signal that varies in time according to at least one of an intrinsic physiological oscillation in the patient and an externally driven oscillation in the patient. For one preferred embodiment, the intrinsic physiological oscillation comprises at least one of an intrinsic respiratory oscillation and a cardiac oscillation. For one preferred embodiment, the externally driven oscillation comprises at least one of an external skull vibrator oscillation and a ventilated respiratory oscillation. The at least one extracted component signal is then processed to generate an output signal representative of the ICP variations in the intracranial compartment.

According to another preferred embodiment, a method for ICP monitoring is provided in which an absolute ICP of a patient is monitored using an invasive ICP monitoring device, such as a subarachnoid bolt. Simultaneously with the invasive ICP monitoring, a non-invasive ICP monitoring device is placed in optical communication with the head of the patient, the non-invasive ICP monitoring device using optical radiation to transcranially detect variations in the magnitudes of periodic intracranial matter oscillations intrinsically and/or extrinsically induced, the magnitude variations being indicative of intracranial matter compliance variations brought about by ICP changes. The absolute ICP from the invasive ICP monitoring device is used to calibrate the non-invasive ICP monitoring device. When the invasive ICP monitoring device is removed, ICP monitoring is continued by maintaining the non-invasive ICP monitoring device in optical communication with the head of the patient.

According to another preferred embodiment, a method for non-invasive ICP monitoring is provided, comprising applying a plurality of discrete mechanical impulses to the head of the patient at a respective plurality of discrete points in time. During each of a plurality of time intervals immediately subsequent to each respective discrete point in time, optical radiation is applied to the patient that propagates transcranially into the intracranial compartment, and optical radiation that has migrated transcranially outward from the intracranial compartment is detected. A plurality of time signals representative of the optical radiation detected during the respective time intervals is then processed to generate an output signal representative of the ICP variations. For one preferred embodiment, the processing comprises, for each of the time signals, computing at least one transient characteristic thereof induced by the mechanical impulse associated therewith. On an impulse over impulse basis, a decreasing value is assigned for the ICP output signal when the computed transient characteristic(s) change toward values indicative of greater intracranial matter compliance, while an increasing value is assigned for the ICP output signal when the computed transient characteristic(s) change toward values indicative of lesser intracranial matter compliance.

### BRIEF DESCRIPTION OF THE DRAWINGS

FIG. 1 illustrates a system for non-invasive monitoring of intracranial pressure (ICP) variations according to a preferred embodiment;

FIG. 2 illustrates conceptual side cutaway views of an intracranial compartment at a valley and a peak, respectively, of the respiratory cycle during an interval in which the ICP is relatively low, along with a corresponding plot of a filtered component of the optically detected signal;

FIG. 3 illustrates conceptual side cutaway views of the intracranial compartment of FIG. 2 at a valley and a peak, respectively, of the respiratory cycle during an interval in which the ICP is relatively high, along with the corresponding plot of the filtered component of the optically detected signal;

FIG. 4 illustrates a system for non-invasive monitoring of intracranial pressure (ICP) variations according to a preferred embodiment;

FIG. 5 illustrates non-invasive monitoring of intracranial pressure (ICP) variations according to a preferred embodiment;

FIG. 6 illustrates a method for ICP monitoring according to a preferred embodiment; and

FIG. 7 illustrates conceptual time plots corresponding to a method for ICP monitoring according to a preferred embodiment.

### DETAILED DESCRIPTION

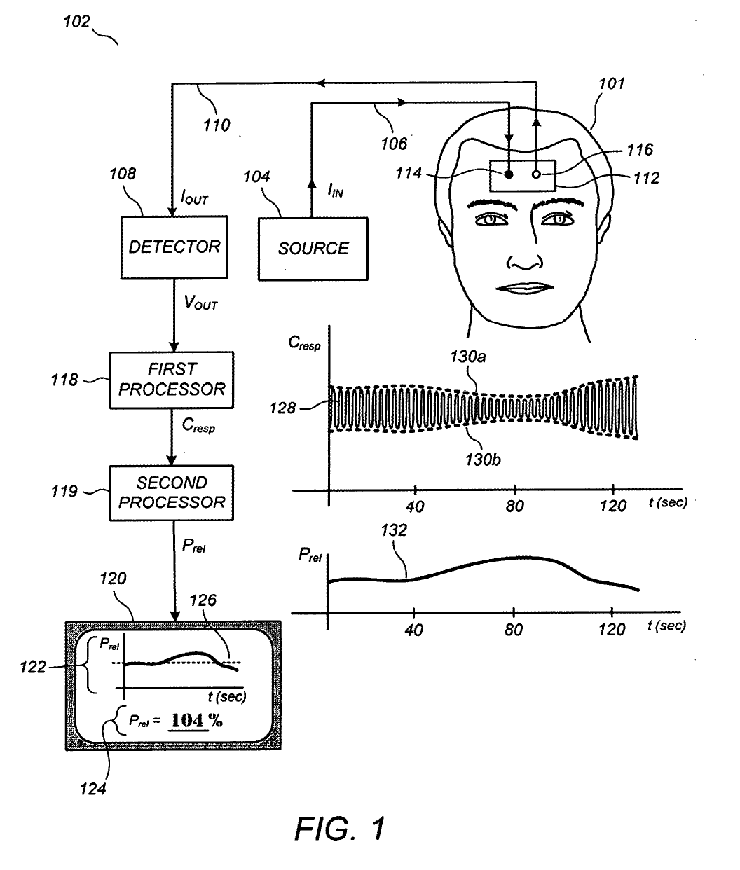

FIG. 1 illustrates a system 102 for non-invasive monitoring of intracranial pressure (ICP) variations of a patient 101 according to a preferred embodiment. Spectrophotometric systems based on visible and/or near infrared (NIR) radiation for achieving various non-invasive physiological measurements, such as transcranial measurements of oxygenated hemoglobin (HbO) and deoxygenated hemoglobin (Hb) concentrations, have been in various stages of proposal and development for an appreciable number of years. As will be appreciated by one skilled in the art in view of the present disclosure, certain component devices suitable for use within the system 102 are described in several prior disclosures directed to such non-invasive optical HbO/Hb measurement, and their specifics will not be re-described here. Moreover, several of those overall spectrophotometric systems and methods may be advantageously used in conjunction with, or as important components within, a system 102 according to one or more of the preferred embodiments. Examples of such spectrophotometric systems include, but are not limited to: continuous wave spectrophotometers (CWS) as discussed in WO1992/20273A2 and WO1996/16592A1; phase modulation spectroscopic (PMS) units as discussed in U.S. Pat. No. 4,972,331, U.S. Pat. No. 5,187,672, and WO1994/21173A1; time resolved spectroscopic (TRS) units as discussed in U.S. Pat. No. 5,119,815, U.S. Pat. No. 5,386,827, and WO1994/22361A1; and phased array systems as discussed in WO1993/25145A1. All of the patents and patent publications identified above in this paragraph are incorporated by reference herein. Another example of a spectrophotometric system that is particularly suitable for use in conjunction with one or more of the preferred embodiments is discussed in US 2006/0015021A1, which is also incorporated by reference herein.

System 102 comprises an optical source 104 that emits radiation having a wavelength in the range of about 500 nm-1000 nm, i.e., in the upper visible and near infrared wavelengths. Light from the optical source 104 is carried by an optical fiber 106 to a source port 114 of an optical coupling device 112 on the forehead of the patient. Light that has migrated through at least a portion of the intracranial compartment and outward again through the cranium is collected at a detection port 116 of the optical coupling device 112 and guided to an optical detector 108 by an optical fiber 110. For one preferred embodiment, the optical coupling device 112 can be similar to one or more of the optical coupling devices disclosed in U.S. Pat. No. 5,596,987, which is incorporated by reference herein. Preferably, the optical coupling device 112 is designed to be a disposable, one-time-use patch that secures to the forehead using known adhesives. The optical coupling device 112 including the source port 114 and detection port 116 can alternatively be attached to an accessible skin surface elsewhere on the scalp other than the forehead.

The detector 108 generates a signal that is representative of the light collected at the detection port 116. For a relatively simple continuous wave embodiment in which the source 104 emits a monochromatic unmodulated carrier wave, the detector 108 provides a voltage signal V_{OUT} representing an instantaneous intensity of the light collected at detection port 116. For one embodiment, the optical source 104 comprises a 4 mW laser diode emitting at 760 nm, and the optical detector 108 comprises a Hamamatsu R928 photomultiplier tube. Although the optical source 104, optical detector 108, and optical coupling device 112 are illustrated as distinct components in the example of FIG. 1, the scope of the present teachings is not so limited. For example, in other preferred embodiments, the optical source(s) and optical detector(s) can be integrated into a single patch that adheres to the skin surface, such that there is no need for external optical connections to the adhesive patch assembly. Any of a variety of other schemes for causing optical radiation to be introduced into the cranium and for causing optical radiation propagating back out of the cranium to be detected can be used without departing from the scope of the preferred embodiments.

As used herein, intracranial compartment refers to the space inside the cranium, while intracranial matter refers broadly to the matter that occupies the intracranial compartment. The intracranial compartment encompasses, and the intracranial matter includes, the dura mater, subdural cavity, arachnoid layer, subarachnoid cavity, pia mater, and brain tissue, along with cerebrospinal fluid contained in the subdural cavity, the blood running throughout to all of the living tissue cells, and the arteries, capillaries, veins, etc., that carry the blood.

As used herein, intrinsic physiological oscillation refers to a physiological characteristic or behavior that is brought about autonomically by the patient's body, that exhibits some form of periodicity, and that directly or indirectly brings about some form of corresponding motion, even if slight, in the intracranial matter of the patient. The corresponding motion can be in the form of positional shifts ranging from very small, localized positional shifts to regional or widespread positional shifts, as well as positional shifts ranging from ordered or patterned positional shifts to disordered or random positional shifts. By way of non-limiting example, as the term positional shift is used herein, intracranial matter that is exhibiting a volume change (e.g., expansion or contraction), whether it be on a local basis or a widespread basis, is also exhibiting a positional shift, since at least some individual portion of that intracranial matter is necessarily moving relative to at least some other individual portion of that intracranial matter as part of that volume change. Likewise, by way of further non-limiting example, as the term positional shift is used herein, a wall of an intracranial artery that is undergoing expansion and contraction as part of a cardiovascular oscillation cycle is also exhibiting positional shifts, since at least some individual portion of that wall is necessarily moving relative to at least some other individual portion of that wall as part of those expansions and contractions.

One example of an intrinsic physiological oscillation is the patient's intrinsic respiratory oscillations, i.e., their natural breathing, which generally occurs at a periodic rate somewhere between 3 breaths per minute (0.05 Hz) and 30 breaths per minute (0.5 Hz). It has been observed that there is some degree of motion, in the form of slight positional shifts/volume changes, in at least a portion of the intracranial matter that occurs in conjunction with the respiratory oscillations of the patient. Another example of an intrinsic physiological oscillation is the patient's cardiac oscillations, which generally occur at a rate somewhere between 30 beats per minute (0.5 Hz) to 180 beats per minute (3 Hz). It has been observed that there is some degree of motion, in the form of slight positional shifts, that occur with the cardiac oscillations (heartbeat) of the patient.

As used herein, externally driven oscillation refers to a physiological characteristic or behavior that is brought about by an external force or input, that exhibits some form of periodicity, and that directly or indirectly brings about some form of corresponding motion, even if slight, in the intracranial matter of the patient. One example of an externally driven oscillation is a ventilated respiratory oscillation that occurs when the patient is placed on a ventilator. Just as with natural breathing, each ventilator-induced breath brings about some degree of positional shift/volume change in at least a portion of the intracranial matter relative to the cranium. However, unlike natural breathing, the operation of a ventilator is at a fixed periodic rate set by an attending clinician. Another example of an externally driven oscillation is an external skull vibrator oscillation brought about by a mechanical vibrator positioned in mechanical communication will the patient's skull. With advantages to be described further hereinbelow, there is provided in one preferred embodiment a non-invasive ICP monitoring system that includes at least one mechanical vibrator operating at a subsonic frequency in the range of about 3 Hz-30 Hz that is positioned so as to vibrate the patient's skull at that rate. Preferably, the intensity of the mechanical vibration is high enough to cause some degree of corresponding motion in at least a portion of the intracranial matter, but gentle enough not to cause too much discomfort to the patient. Toward this end, the duty cycle of the mechanical vibrator can be restricted to being “on” for only a few seconds, perhaps 4-5 seconds, every two or three minutes, and “off” otherwise.

As may be evident from the incorporated references, the particular physics and mathematics of the scattering and attenuation of the light as it propagates in a banana-shaped migration path from the source port 114 to the detection port 116 can be quite complicated, even when various simplifying assumptions are made regarding the various bone, tissue, and fluid types traversed. However, in accordance with a preferred embodiment, ICP variations are detected in a relatively elegant manner that transcends the particular scheme (CWS, PMS, TRS, etc.) by which the interrogating light waves are modulated, introduced, detected, and evaluated. As described above, it has been observed that at least a portion of the intracranial matter will experience some type of periodic motion relative to the cranium, in the form of positional shift and/or volume change, in correspondence with the above-described intrinsic physiological oscillations. Alternatively or in conjunction therewith, at least some periodic motion of the intracranial matter can be induced in correspondence with externally driven oscillations. Furthermore, it has been found that the amount of this periodic motion will become more restricted at higher ICP pressures and less restricted at lower ICP pressures. If a signal is extracted from the detected radiation that varies in magnitude (or other measurable amount) with an intrinsic physiological oscillation or an externally driven oscillation, then that extracted signal can be used to detect ICP variations regardless of the designed physiological significance (if any) of that extracted signal. Generally speaking, larger variations in that extracted signal will be indicative of a lower ICP, because the intracranial matter is less restricted in its periodic motion when the ICP is lower. Likewise, smaller variations in the extracted signal will be indicative of a higher ICP, because the intracranial matter is more restricted in its periodic motion when the ICP is higher.

For one preferred embodiment, a single signal is extracted from the detected radiation that varies in magnitude (or other measurable amount) with a single intrinsic physiological oscillation or a single externally driven oscillation. For another preferred embodiment, multiple signals are extracted from the detected radiation that vary in magnitude (or other measurable amount) with multiple respective intrinsic physiological oscillations, multiple respective externally driven oscillations, or a combination of at least one respective intrinsic physiological oscillation and at least one respective externally driven oscillation.

FIG. 1 illustrates an example of the preferred embodiment in which only a single signal is extracted from the detected radiation, wherein that signal varies in magnitude with the intrinsic respiratory oscillations of the patient. Provided in accordance with this preferred embodiment is a first processor 118 (which can alternatively be analog filter circuit) that processes the signal V_{OUT} in digital form to extract therefrom a component signal Cᵣₑₛₚ that varies in time according to a timewise respiratory pattern of the patient. Illustrated in FIG. 1 is a conceptual plot 128 of the component signal Cᵣₑₛₚ, which varies cyclically within an envelope 130a/130b. Any of a variety of known filtering methods can be used, ranging from a simple numerical digital filter having a passband at typical breathing rates (e.g. between 0.05 Hz and 0.5 Hz), to more complex lock-in schemes using a reference signal from a respiration transducer (not shown), such as a Pneumotrace™ respiration transducer model TSD101 from BIOPAC Systems, Inc., of Goleta, Calif. Optionally, the optical source 104 can be tuned for different wavelengths such that an optimal radiation wavelength (i.e., the radiation wavelength for which the most pronounced and ICP-sensitive component signal Cᵣₑₛₚ is obtained) can be identified by the user. Alternatively, laboratory tests can be run to determine a best predetermined wavelength.

Also provided in accordance with this preferred embodiment is a second processor 119 (which can alternatively be analog filter circuit, and which can optionally be integral with the first processor 118) that processes the component signal Cᵣₑₛₚ in digital form to provide an output signal Pᵣₑₗ indicative of the ICP variations in the intracranial compartment. As part of the processing by the second processor 119, an envelope magnitude (i.e., the vertical distance between the plot lines 130a and 130b) of the component signal Cᵣₑₛₚ is determined. The output signal Pᵣₑₗ is assigned a greater value when the envelope magnitude has a lesser value, and the output signal Pᵣₑₗ is assigned a lesser value when the envelope magnitude has a greater value. System 102 further comprises a user display 120 providing a graphical representation 122 and/or a numerical representation 124 of the ICP output value Pᵣₑₗ as a percentage of a baseline value 126.

It is to be appreciated that envelope magnitude (i.e., the vertical distance between upper and lower envelope lines) represents one of a variety of different amplitude characteristics of the component signal Cᵣₑₛₚ that can be measured and used in the determination of the output signal Pᵣₑₗ without departing from the scope of the present teachings. More generally, any amplitude characteristic of the component signal Cᵣₑₛₚ (i.e., any metric that characterizes an AC strength of the component signal Cᵣₑₛₚ) may be used in place of the envelope magnitude, such as an RMS value, a time average of a rectified version, a standard deviation, a square (or cube, etc) of the peak-to-peak value, and so on, without departing from the scope of the preferred embodiments. Thus, descriptions provided herein with respect to envelope magnitude of the component signal Cᵣₑₛₚ, which are provided for purposes of clarity of presentation, are applicable for other amplitude characteristics of the component signal Cᵣₑₛₚ as well.

The particular nature of the inverse relationship between the envelope magnitude of the component signal Cᵣₑₛₚ and the output value Pᵣₑₗ (e.g., whether it is a reciprocal relationship, a negative arithmetic relationship, or other inverse relationship) could be determined empirically based on test scenarios by a person skilled in the art without undue experimentation in view of the present disclosure. By way of example, a set of test data can be developed in clinical data-gathering trials by applying the system 102 to a population of patients during periods in which their absolute ICP levels are being monitored by an invasive ICP monitoring device, such as a subarachnoid bolt, which is currently recognized as the “gold standard” in ICP measurement. The outcome of the clinical data-gathering trials can be used to establish a relationship between (i) the percentage of envelope magnitude change from an initial envelope magnitude baseline, and (ii) the percentage of ICP variation from the corresponding initial absolute ICP reading. This can then be used to provide the ICP output value Pᵣₑₗ as a percentage of the baseline value 126. Depending on the results of the clinical data-gathering trials, it may be possible to establish a set of normative data based on different patient characteristics (e.g., height, weight, body surface area to weight ratio, etc.) to provide a more precise mapping between percent envelope magnitude change and percent ICP change. Indeed, it may even be possible, and would certainly be within the scope of the preferred embodiments, to establish a set of normative data that allows absolute ICP levels to be computed based on certain patient information as combined with the envelope magnitude changes and/or envelope magnitude levels, in which case the Pᵣₑₗ output shown in FIG. 1 would be replaced by a P_{abs} output expressed in mm Hg.

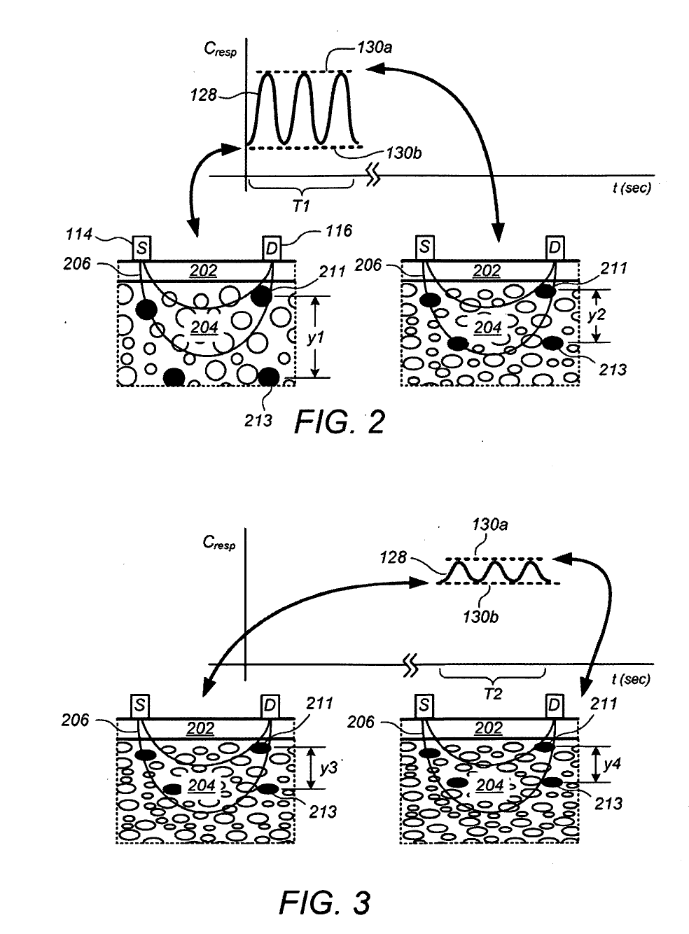

FIG. 2 illustrates conceptual side cutaway views of an intracranial compartment 204 at a valley (left side) and a peak (right side) of respiratory oscillations during an interval T1 in which the ICP is relatively low. It is to be appreciated that although the example of respiratory oscillations is presented for clarity of disclosure in correspondence with the example of FIG. 1, supra, similar conceptual illustrations apply for other types of intrinsic physiological oscillations and externally driven oscillations. Notably, the terms “valley” and “peak” as used herein do not necessarily represent any particular phase of the respiratory cycle, such as inhale or exhale, but instead simply represent extremes of the intracranial matter motion that occurs during the respiratory cycle, whenever those extremes might occur. Also shown is a corresponding plot 128 of the component signal Cᵣₑₛₚ across three respiratory cycles. Also illustrated is the cranial bone 202 (the skin above the cranial bone is omitted), the source port 114, and the detection port 116. The optical radiation migrates through a generally banana-shaped path 206 between the source port 114 and the detection port 116. The intracranial compartment 204 includes intracranial matter that is represented conceptually by arbitrarily encircled sections, with four arbitrary ones of the encircled sections being colored black for easier recognition including the sections 211 and 213.

During the peak (right side) of a respiratory cycle, the intracranial matter is deformed toward the cranial bone 202 by a slightly greater amount than during the valley (the drawings are exaggerated for clarity). Thus, for example, there is a greater distance y1 between sections 211 and 213 during the valley (left side) and a lesser distance y2 during the peak (right side). It is these slight shifts of the intracranial matter that cause the variations of the detected optical signal as extracted at the respiration frequency range. Notably, although it is believed that much of the intracranial matter shifting is due to subdural cavity deformation between the dura mater and arachnoid layers, the true physiological nature of the deformation (e.g., which tissues are deforming by what amount, is the deformation conformal versus irregular, etc.) is generally irrelevant for the purposes of measuring the ICP variations in accordance with the preferred embodiments. Rather, the main requirement is simply that “something” is deforming, in “some” manner that affects the detected optical signal in “some” measurable way according to the respiration cycle of the patient.

FIG. 3 illustrates the intracranial compartment 204 at a valley (left side) and a peak (right side) of the respiratory cycle during an interval T2 in which the ICP is relatively high. As indicated by a lesser difference (y3−y4) than in FIG. 2 between the valley and peak positions of the sections 211 and 213, there is less deformation between the valleys and peaks due to the greater ICP level.

As used herein, compliance refers to the property of intracranial matter that is illustrated in the examples of FIG. 2 and FIG. 3, that is, the degree of corresponding periodic motion, in the form of positional shifts and/or volume changes, in all or a portion of the intracranial matter as a result of an intrinsic physiological oscillation (such as a respiratory oscillation as used in the above examples) or an externally driven oscillation. When the ICP is lower, the compliance of the intracranial matter increases. When the ICP is higher, the compliance of the intracranial matter decreases. Thus provided in accordance with one aspect of the present teachings is a non-invasive ICP measuring device that uses optical radiation to transcranially detect variations in the magnitudes of periodic intracranial matter oscillations that are intrinsically and/or extrinsically induced, the magnitude variations being indicative of intracranial matter compliance variations brought about by ICP changes.

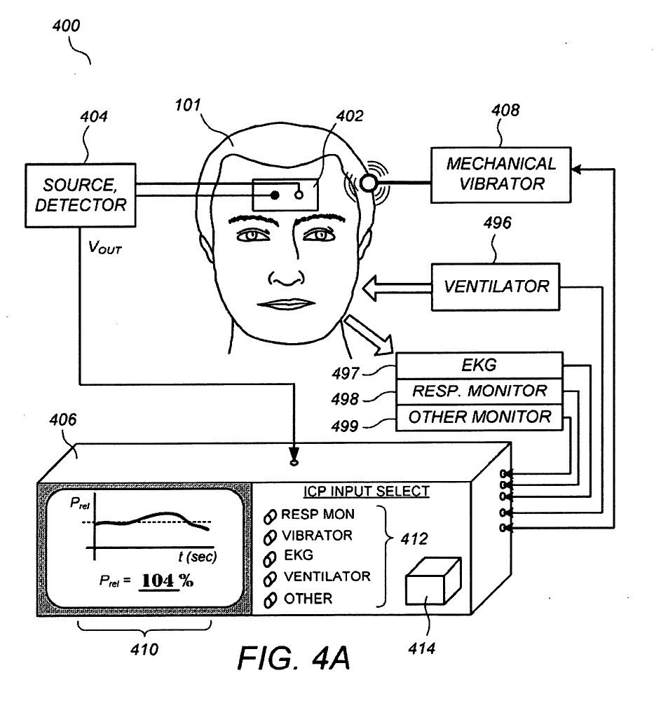

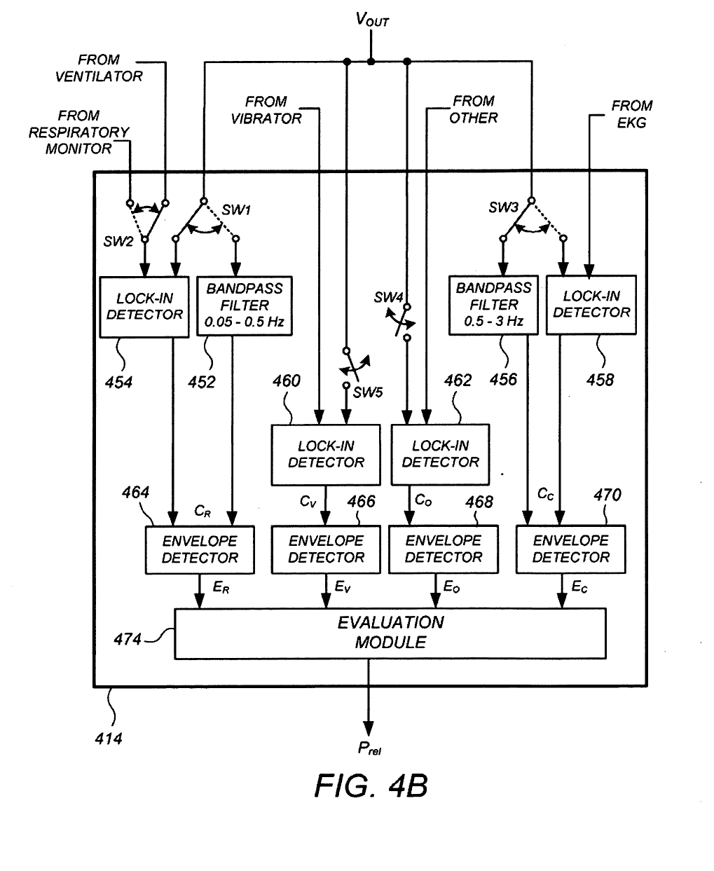

FIGS. 4A illustrates a system 401 for non-invasive monitoring of intracranial pressure (ICP) variations of a patient 101 according to a preferred embodiment in which multiple signals are extracted from the detected radiation that vary in magnitude (or other measurable amount) with multiple respective intrinsic physiological oscillations, multiple respective externally driven oscillations, or a combination of at least one respective intrinsic physiological oscillation and at least one respective externally driven oscillation. For one preferred embodiment, each of the multiple signals is separately filterable (or otherwise extractable) from the detected radiation by virtue of a distinct set of frequencies occupied by its underlying intrinsic physiological oscillation or externally driven oscillation. Upon extraction, each of the extracted signals is individually processed to determine an intracranial matter compliance metric, such as the envelope magnitude, corresponding to the underlying intrinsic physiological oscillation or externally driven oscillation.

Generally speaking, all of the intracranial matter compliance metrics (e.g., envelope magnitudes) will share a common characteristic in that each will generally increase as the ICP decreases, and that each will generally decrease as the ICP increases. However, it has been found that a rich variety of clinically interesting and relevant information can arise from the fact that these different intracranial matter compliance metrics (e.g., envelope magnitudes) will generally exhibit different differential characteristics with changing ICP as a function of the prevailing absolute level of ICP. By way of example, letting the variable E_{R} represent the respiratory intracranial matter compliance metric (e.g., envelope magnitude of the extracted respiratory component of the detected optical signal), and letting the variable E_{C} represent the cardiac intracranial matter compliance metric (e.g., envelope magnitude of the extracted cardiac component of the detected optical signal), it has been found that E_{R} tends to diminish rapidly with increasing ICP when the absolute ICP is at moderate levels. However, as the absolute ICP increases further, E_{R} tends asymptotically toward zero, such that at high levels of absolute ICP, E_{R} metric ceases to change in any measurable way with increased ICP. In contrast, the cardiac envelope E_{C} tends to be quite robust against increases in absolute ICP, and maintains appreciable nonzero values even for high levels of absolute ICP. In accordance with a preferred embodiment, both of the metrics E_{R} and E_{C} are computed from the detected signal information, and their values relative to each other are analyzed (such as by taking their ratio, difference, etc.) to yield increased precision in the ICP determination process and/or to derive other useful information regarding the health of the patient. The specific ways in which E_{R} and E_{C} can be advantageously processed can be determined, for example, by using data from a large clinical data-gathering trial, where E_{R} and E_{C} are tracked along with absolute ICP and other vital signs, and patterns and/or statistical correlations in the data can be developed. Indeed, it would not be outside the scope of the preferred embodiments for a set of normative data to be developed using multivariate correlations among E_{R}, E_{C}, E_{V} (e.g., envelope magnitude of the extracted subsonic vibratory component of the detected optical signal), and other intracranial matter compliance metrics such that the non-invasive ICP monitoring device can be automatically calibrated based on these computed values for providing absolute ICP level determinations.

Thus, provided in the system 401 according to a preferred embodiment is an optical coupling patch 402 and source/detector system 404 for providing a voltage signal V_{OUT} representing an instantaneous intensity of light collected at a detection port of the optical coupling patch 402, in a manner similar to like elements of FIG. 1, supra. System 401 further comprises a console 406 comprising an output display 410 similar to the output display 120 of FIG. 1, supra, a user input device 412, and a processor 414 configured and programmed to perform the functionalities described further herein. System 401 further comprises a mechanical vibrator 408 configured to apply a subsonic mechanical vibration to the skull of the patient 101. Also shown in FIG. 4A is various external instrumentation equipment that is commonly available in a clinical setting, including a ventilator 496, an EKG monitor 497, a respiratory monitor 498, and “other” device 499 that is capable of inducing and/or measuring some other form of intrinsic physiological oscillation or externally driven oscillation. The console 406 is coupled to receive V_{OUT} from the source/detector 404, to receive a vibration frequency from the mechanical vibrator 408 (or to dictate such frequency to the mechanical vibrator 408), to receive a ventilation frequency or signal pattern from the ventilator 496, to receive EKG signals from EKG monitor 497, to receive respiratory signals from respiratory monitor 498, and “other” signals from “other” monitor 499.

Notably, many different combinations of the above-described elements 408, 496, 497, 498, and 499 can be hooked up to the console 406 without departing from the scope of the preferred embodiments, including an option in which none of them are hooked up and only the signal V_{OUT} is provided to the console. Generally speaking, as more normative clinical data is gathered, the selected ones of these hookups providing the most useful signals will be identified, and increasingly precise results, even up to and including calibrated absolute ICP measurements, can be obtained. However, even in a simplest embodiment in which no external hookups are provided except for V_{OUT}, the system 401 is still useful as an indicator as to whether the ICP is increasing, decreasing, or staying the same. Preferably, the processor 414 is configured to be easily upgradable, such as by firmware flash or internet download, so that the latest and best capabilities are integrated as more and more normative clinical data is gathered.

The user input device 412 allows a user, such as a clinician, to select the basis upon which non-invasive ICP measurement is to be made. Depending upon which buttons the user selects, the processor 414 will “listen” to the appropriate external signals, extract the relevant components from V_{OUT}, and provide a best estimate Pᵣₑₗ (or, potentially, P_{absolute}) for display to the clinician.

FIG. 4B illustrates a schematic diagram of the processor 414, which can be implemented in any of a variety of physical configurations (e.g., in software general purpose processor, in hardware on application specific integrated circuit (ASIC), various combinations thereof, etc.) without departing from the scope of the preferred embodiments. Processor 414 comprises a bandpass filter 452 that is designed to extract a respiratory component C_{R} in a manner similar to the first processor 118 of FIG. 1, supra. The bandpass filter 452 is selected at switch SW1 if the user has chosen neither the ventilator input nor the respiratory monitor input on the input device 412. However, if the user has selected the ventilator or respiratory monitor option, then a lock-in detector 454 is selected at switch SW1, with a reference signal being from either the ventilator or respiratory monitor input via switch SW2 per the user's selection.

As used herein, lock-in detector refers to a device or algorithm that receives an input signal and a periodic reference signal, and synchronously extracts frequency components from the input signal that correspond to the frequency content of the periodic reference signal. Generally speaking, if a periodic reference signal is available, lock-in detection is highly superior to passive bandpass filtering with respect to signal-to-noise performance, and so the processor 414 generates the signal C_{R} using the bandpass filter 452 as a “last resort” when the user has chosen neither the ventilator nor the respiratory monitor. However, the scope of the preferred embodiments is not so limited, and in other preferred embodiments, plural versions of the C_{R} signal can be generated using both the lock-in detector 454 and bandpass filter 452, and both versions can be considered as distinct inputs to the evaluation module after envelope detection. It still another preferred embodiment, three versions of the C_{R} signal can be created, including one version from the bandpass filter 452, a second version from the lock-in detector 454 using the ventilator reference signal, and a third version from the lock-in detector 454 using the respiratory monitor reference signal.

The signal C_{R}, which is analogous to the periodic component signal Cᵣₑₛₚ of FIG. 1, supra, at plot 128, is then fed to an envelope detector 464 for extracting the envelope signal E_{R}, which is analogous to the distance between the envelopes 130a/130b of the plot 128 of FIG. 1. As discussed previously, the envelope signal E_{R} represents a measure of the intracranial matter compliance with respect to the respiratory oscillations of the patient. In another preferred embodiment (not shown), there is an option to turn off the respiratory channel entirely, in which case neither bandpass filter 456 nor the lock-in detector 458 is active and no respiratory component is input to the evaluation module 474.

Processor 414 further comprises a bandpass filter 456 that is designed to extract a cardiac component C_{C} from the signal V_{OUT}. The bandpass filter 456 is selected at switch SW3 if the user has not chosen the EKG signal on the input device 412. However, if the user has indeed selected the EKG signal, then a lock-in detector 458 is selected at switch SW3, with a reference signal being from the EKG output. The signal C_{C} is then fed to an envelope detector 470 for extracting the envelope signal E_{C}, which represents a measure of the intracranial matter compliance with respect to the cardiac oscillations of the patient. In another preferred embodiment (not shown), there is an option to turn off the cardiac channel entirely, in which case neither bandpass filter 456 nor the lock-in detector 458 is active and no cardiac component is input to the evaluation module 474.

Processor 414 further comprises a lock-in detector 460 that is designed to extract an externally driven vibratory component C_{V} from the signal V_{OUT}. There is generally no need for a passive bandpass filter here because a reference signal should always be available, although the scope of the preferred embodiments is not so limited. The signal C_{V} is then fed to an envelope detector 466 for extracting the envelope signal E_{V}, which represents a measure of the intracranial matter compliance with respect to the externally driven subsonic vibratory oscillations of the patient. The switch SW5 is opened to turn off the subsonic vibratory oscillation channel if the user has not selected it on the input device 412.

Processor 414 further comprises a lock-in detector 462 that is designed to extract an “other” oscillatory component C_{O} from the signal V_{OUT}. Generally speaking, there may be a variety of other periodic inputs that could lead to corresponding intracranial matter oscillations, including those that are not yet currently known. By way of somewhat fanciful example, large periodic doses of therapeutic radiation might someday be applied that cause corresponding intracranial matter oscillations. The extraction of such “other” oscillatory components from the signal V_{OUT} and processing them to detect a metric of corresponding intracranial compliance is not outside the scope of the preferred embodiments. As illustrated in FIG. 4B, the signal C_{O} is then fed to an envelope detector 468 for extracting the envelope signal E_{C}, which represents such metric of corresponding intracranial compliance. The switch SW4 is opened to turn off the “other” oscillation channel if the user has not selected it on the input device 412.

Finally, evaluation module 474 receives those of E_{R}, E_{V}, E_{O}, and E_{C} that are available according to the user's input and computes therefrom the output Pᵣₑₗ (or, potentially, P_{absolute}) for display on the display output 410. Similar to the discussion supra with respect to FIG. 1, the particular algorithm by which a useful value for Pᵣₑₗ will be calculated can be determined, and continually improved, as further clinical data-gathering trials are completed and optimal statistical relationships determined. In one simple example, the percentage change in each of E_{R}, E_{V}, E_{O}, and E_{C}, and some average thereof, is monitored, and an output is provided that is assigned a decreasing value as that average increases and that is assigned an increasing value as that average decreases. Optionally, any of a variety of other outputs based on E_{R}, E_{V}, E_{O}, or E_{C} can be provided in accordance with the gathered normative data.

It is to be appreciated that the scope of the preferred embodiments is not limited to the continuous wave scenario of FIGS. 1 and 4A-4B. In another preferred embodiment (not shown), the emitting and detecting performed by the source(s) and detector(s) can be in accordance with phase modulation spectroscopy (PMS) or time resolved spectroscopy (TRS) principles, provided only that a one-dimensional signal (e.g., a time-varying voltage) representative of the detected output radiation (e.g. phase shift, time of flight, etc.) is provided to the first processor 118 (FIG. 1) or processor 414 (FIG. 4B) that is at least partially dependent upon the intrinsic physiological oscillation(s) and/or an externally driven oscillation(s) in the patient.

In yet another preferred embodiment, (not shown), plural arrays of sources and detectors can be positioned and operated according to CWS, PMS, TRS, or other principles such that a two-dimensional map or image of a spatially varying property within the intracranial compartment is generated, the two dimensional image being time-varying and morphing, even if slightly so, according to the intrinsic physiological oscillation(s) and/or an externally driven oscillation(s) in the patient. Image processing can then be performed on the time-varying image to generate a metric related to an amount of morphing that is happening in correspondence with those oscillations. In one simple example, the amount of morphing can be identified as the time-varying distance between two landmark locations in the two-dimensional image. This metric can then be treated like the voltage V_{OUT} in FIG. 1 or FIG. 4, supra, and the ICP variations can be computed therefrom as previously described. Notably, the particular physiological significance of the two-dimensional image (e.g., an oxygenation map, attenuation map, scattering map) will usually not be as important as the fact that it morphs measurably and in conjunction with the intrinsic physiological oscillation and/or externally driven oscillation in the patient. Advantageously, however, the two-dimensional image could be used for other useful purposes in conjunction with its use as a basis for ICP monitoring.

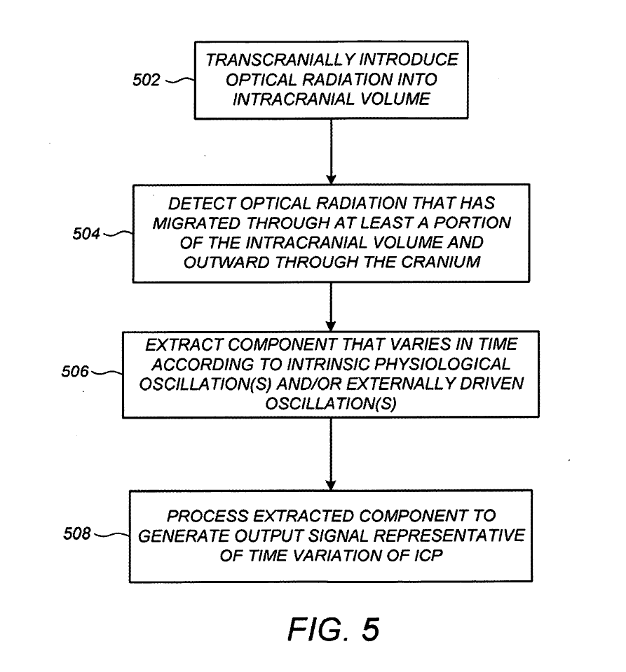

FIG. 5 illustrates non-invasive monitoring of ICP variations according to a preferred embodiment. At step 502, optical radiation is introduced transcranially into the intracranial compartment. At step 504, optical radiation is detected that has migrated through at least a portion of the intracranial compartment and back outward through the cranium. At step 506, at least one signal representative of the detected optical radiation is processed to extract therefrom a component signal that varies in time according to one or more intrinsic physiological oscillations and/or one or more externally driven oscillations in the patient. Finally, at step 508, the extracted component signal is processed to generate therefrom an output signal representative of the ICP variations in the intracranial compartment.

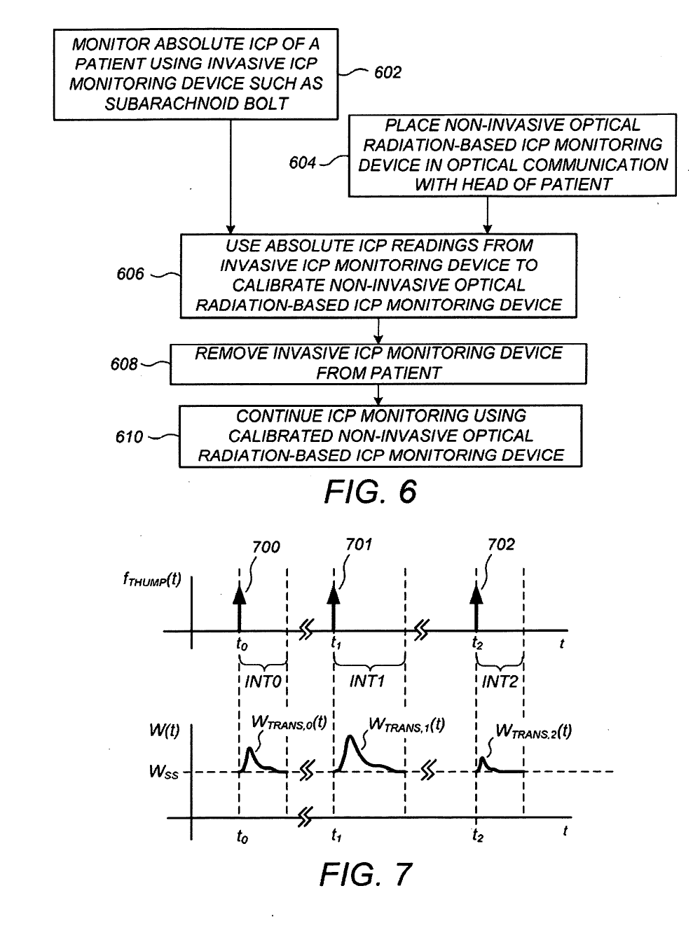

FIG. 6 illustrates a method for ICP monitoring in accordance with a preferred embodiment. At step 602, an absolute ICP of a patient is monitored using an invasive ICP monitoring device such as a subarachnoid bolt. Although invasive ICP monitoring devices such as subarachnoid bolts are the gold standard for ICP measurement, their use can bring about infection or other negative consequences when left in the patient's skull for too long a period of time. According to a preferred embodiment, at step 604, a non-invasive ICP monitoring device is placed in optical communication with the head of the patient while the invasive ICP monitoring device is still in the patient's skull. Preferably, the non-invasive ICP monitoring device uses optical radiation to transcranially detect variations in the magnitudes of periodic intracranial matter oscillations intrinsically and/or extrinsically induced, the magnitude variations being indicative of intracranial matter compliance variations brought about by ICP changes. At step 606, the absolute ICP from the invasive ICP monitoring device is used to calibrate the non-invasive ICP monitoring device At a minimum, this can be used to establish a baseline output reading for the non-invasive unit in absolute mm Hg, for cases in which the patient's ICP remains constant during the simultaneous monitoring. On the other hand, if the patient's ICP fluctuates during simultaneous monitoring, a more complete multi-point calibration of the non-invasive unit can be achieved that will be accurate at least within the range of fluctuation that has occurred, and possibly beyond that range if normative data from clinical data-gathering trials dictates that some degree of extrapolation can safely occur. At step 608, the ICP monitoring device is removed, which can be triggered by the normal course of a therapeutic intervention, or which alternatively be triggered by a determination that sufficient calibration of the non-invasive ICP monitor has been achieved. Finally, at step 610, ICP monitoring is continued by maintaining the non-invasive ICP monitoring device in optical communication with the head of the patient.

FIG. 7 illustrates conceptual time plots corresponding to a method for ICP monitoring according to another preferred embodiment in which an “impulse response” of the intracranial matter, as measured by a transient effect on the detected optical signal(s) induced by a discrete mechanical impulse on the head of the patient, is monitored over time. For this embodiment, the mechanical vibrator 408 of FIG. 4, supra, is replaced by a mechanical thumper (not shown). The mechanical thumper can be, for example, a pre-calibrated spring-loaded plunger that delivers known impulses (force thumps) to the skull of the patient, or another type of mechanical transducer having similar effect. The mechanical thumper can operate in a recoil-based manner (analogous to a recoil hammer that bounces back after striking) or in a non-recoil-based manner (analogous to a deadblow hammer that does not bounce back after striking) without departing from the scope of the preferred embodiments.

Referring again to FIG. 7, using the mechanical thumper, a plurality of discrete mechanical impulses 700, 701, and 702 are applied to the head of the patient at a respective plurality of discrete points in time t₀, t₁, and t₂. The time spacing among the time points t₀, t₁, and t₂ can be on the order of seconds or minutes and is not required to be constant, although the scope of the preferred embodiments is not so limited. Indeed, the time between impulses can even be dynamically variable, for example, at reduced intervals when the ICP is varying relatively quickly with time.

During each of a plurality of time intervals (INT0, INT1, INT2) immediately subsequent to each respective discrete point in time (t₀, t₁, t₂) optical radiation is applied to the patient that propagates transcranially into the intracranial compartment, and optical radiation that has migrated transcranially outward from the intracranial compartment is detected. A plurality of time signals (W_{TRANS,0}(t), W_{TRANS,1}(t), W_{TRANS,1}(t)) representative of the optical radiation detected during the respective time intervals (INT0, INT1, INT2) is then processed to generate an output signal representative of the ICP variations.

For one preferred embodiment, the processing comprises, for each of the time signals (W_{TRANS,0}(t), W_{TRANS,1}(t), W_{TRANS,1}(t)), computing at least one transient characteristic thereof induced by the mechanical impulse (700, 701, 702, respectively) associated therewith. Preferably, on an impulse over impulse basis, a decreasing value is assigned for the ICP output signal when the computed transient characteristic(s) change toward values indicative of greater intracranial matter compliance, while an increasing value is assigned for the ICP output signal when the computed transient characteristic(s) change toward values indicative of lesser intracranial matter compliance. For a particular time signal W_{TRANS,j}(t), examples of transient characteristics can be the peak difference between W_{TRANS,j}(t) and the steady state value W_{SS} (i.e., the value or characteristic when there has been no thumping for a substantial time), the time-to-peak or rise time after the impulse, the overall time center of mass of the curve W_{TRANS,j}(t), the relaxation time between the peak value at the steady-state value, or any of a variety of other transient characteristics that characterize how much and/or how fast the intracranial matter is shaking, shifting, etc. responsive to the mechanical thumping. Generally speaking, the best type of optical modulation/filtering scheme used to derive W_{TRANS,j}(t), the type and degree of thumping, the particular selection and/or combinations to transient characteristics to compute, the particular manner in which those values are calibrated to relative or absolute ICP metrics, and other relevant factors could be determined by a person skilled in the art (e.g., empirically using structured clinical experiments) in view of the present disclosure without undue experimentation.

Whereas many alterations and modifications of the preferred embodiments will no doubt become apparent to a person of ordinary skill in the art after having read the foregoing description, it is to be understood that the particular embodiments shown and described by way of illustration are in no way intended to be considered limiting. Thus, reference to the details of the described embodiments are not intended to limit their scope.

What is claimed is:

1. A method for non-invasive detection of intracranial pressure (ICP) variations in an intracranial compartment of a patient, comprising:
emitting optical radiation from at least one light source positioned relative to the patient such that at least a portion of the emitted optical radiation migrates transcranially into the intracranial compartment;
detecting, by at least one detector, optical radiation that has migrated through at least a portion of the intracranial compartment and has migrated transcranially outward therefrom;
processing at least one signal representative of said detected optical radiation to extract therefrom at least one component signal that varies in time according to at least one of an intrinsic physiological oscillation in the patient and an externally driven oscillation in the patient; and
processing said at least one extracted component signal to generate therefrom an output signal representative of the ICP variations in the intracranial compartment.

2. The method of claim 1, wherein said at least one intrinsic physiological oscillation comprises at least one of an intrinsic respiratory oscillation and a cardiac oscillation.

3. The method of claim 1, wherein said externally driven oscillation comprises a ventilated respiratory oscillation.

4. The method of claim 1, further comprising bringing an external mechanical vibrator into mechanical coupling with the head of the patient, wherein said externally driven oscillation is induced by said external mechanical vibrator.

5. The method of claim 4, wherein said external mechanical vibrator oscillates at a subsonic frequency between about 3 Hz and 30 Hz.

6. The method of claim 1, wherein said emitted optical radiation is an unmodulated, substantially monochromatic carrier wave having a wavelength within the range of 500 nm-1000 nm.

7. The method of claim 6, wherein said at least one signal representative of said detected optical radiation is a one-dimensional signal representative of an optical intensity of the migrated optical radiation, and wherein said processing to extract said at least one component signal comprises:
extracting a respiratory component signal from said optical intensity signal, said respiratory component signal having a first relatively narrow frequency range corresponding to a respiratory rate of the patient; and
extracting a cardiac component signal from said optical intensity signal, said cardiac component signal having a second relatively narrow frequency range corresponding to a heart rate of the patient.

8. The method of claim 7, wherein said extracting a respiratory component comprises one of bandpass filtering to said first relatively narrow frequency range and lock-in detection using a reference signal comprising an externally provided respiratory signal.

9. The method of claim 8, wherein said externally provided respiratory signal is provided by one of a ventilator and a respiration monitor.

10. The method of claim 7, wherein said extracting a cardiac component comprises one of bandpass filtering to said second relatively narrow frequency range and lock-in detection using a reference signal comprising an externally provided cardiac signal.

11. The method of claim 7, wherein said processing said at least one extracted component signal to generate said output signal comprises:
detecting a first amplitude characteristic of said extracted respiratory component;
detecting a second amplitude characteristic of said extracted cardiac component; and
assigning a value for said output signal based on at least one of said first amplitude characteristic, said second amplitude characteristic, and a comparison between said first amplitude characteristic and said second amplitude characteristic.

12. The method of claim 1, further comprising bringing an external mechanical vibrator into mechanical coupling with the head of the patient, wherein said externally driven oscillation is induced by said external mechanical vibrator, wherein said at least one signal representative of said detected optical radiation is a one-dimensional signal representative of an optical intensity of the migrated optical radiation, wherein said processing to extract said at least one component signal comprises synchronously extracting an externally induced vibration component from said optical intensity signal using a timing signal of said external mechanical vibrator as a reference frequency.

13. The method of claim 12, wherein said processing said extracted component signal to generate said output signal comprises:
detecting an amplitude characteristic of said externally induced vibration component;
assigning a decreasing value for said output signal as said amplitude characteristic increases; and
assigning an increasing value for said output signal as said amplitude characteristic decreases.

14. The method of claim 1, wherein said at least one extracted component signal consists of a single component signal corresponding to a single intrinsic physiological oscillation in the patient or a single externally driven oscillation in the patient, and wherein said processing said at least one extracted component signal to generate said output signal comprises:
detecting an amplitude characteristic of said single component signal;
assigning a decreasing value for said output signal as said amplitude characteristic increases; and
assigning an increasing value for said output signal as said amplitude characteristic decreases.

15. The method of claim 14, wherein said single component signal corresponds to one of an intrinsic respiratory oscillation in the patient, a cardiac oscillation in the patient, a ventilated respiratory oscillation in the patient, and an oscillation induced by an external mechanical vibrator coupled to the head of the patient.

16. The method of claim 1, wherein said emitting and detecting is performed according to one of continuous wave spectroscopy (CWS), phase modulation spectroscopy (PMS) and time resolved spectroscopy (TRS), and wherein said at least one signal representative of said detected optical radiation is a one-dimensional time-varying intensity signal corresponding to an intensity of the received optical radiation.

17. The method of claim 1, wherein said emitting and detecting is performed such that said at least one signal representative of said detected optical radiation is a time-varying two-dimensional image, and wherein said processing to extract said at least one component signal therefrom comprises:
identifying at least two landmark locations in said morphing image that oscillate toward and away from each other at a rate corresponding to the intrinsic physiological oscillation and/or externally driven oscillation underlying the component signal to be detected; and
setting the component signal proportional to the instantaneous separation between said two landmark locations in said time-varying two-dimensional image.

18. The method of claim 1, further comprising:
establishing a baseline value for said output signal according to historical determinations thereof for the patient; and
graphically or numerically displaying said output value on a display device formatted as a percentage of said baseline value.

19. A system for non-invasively detecting intracranial pressure (ICP) variations in an intracranial compartment of a patient, comprising:
a receiving device for receiving at least one signal representative of optical radiation that has migrated transcranially outward from the intracranial compartment after having been transcranially introduced thereinto; and
a processor configured to process said at least one signal to generate therefrom an output signal representative of said ICP variations, wherein said processing said at least one signal comprises (i) extracting therefrom at least one component signal varying in time according to one of an intrinsic physiological oscillation in the patient and an externally driven oscillation in the patient, and (ii) computing said output signal based at least in part on an amplitude characteristic of each of said extracted component signals.

20. The system of claim 19, further comprising:
an optical source disposed in optical communication with the patient such that at least a portion of optical radiation emitted therefrom migrates transcranially into the intracranial compartment;
an optical detector positioned and configured to detect the optical radiation migrating transcranially outward from the intracranial compartment; and
a modulation/demodulation system coupled to said optical source and said optical detector and providing said at least one signal to said receiving device;
wherein said optical source, said optical detector, and said modulation/demodulation system are configured for one of continuous wave spectroscopic (CWS), phase modulation spectroscopic (PMS) and time resolved spectroscopic (TRS) operation.

21. The system of claim 19, wherein said at least one extracted component signal consists of a single component signal corresponding to a single intrinsic physiological oscillation in the patient or a single externally driven oscillation in the patient, and wherein said computing said output signal comprises:
detecting an amplitude characteristic of said single component signal;
assigning a decreasing value for said output signal as said amplitude characteristic increases; and
assigning an increasing value for said output signal as said amplitude characteristic decreases.

22. The system of claim 21, wherein said single component signal varies in time according to one of an intrinsic respiratory oscillation, a cardiac oscillation, and a ventilated respiratory oscillation.

23. The system of claim 21, further comprising an external mechanical vibrator in mechanical communication with the head of the patient, wherein said single component signal varies in time according to an oscillation frequency of said external mechanical vibrator.

24. The system of claim 23, wherein said oscillation frequency of said external mechanical vibrator is between about 3 Hz and 30 Hz.

25. The system of claim 19, further comprising a display device coupled to said processor for displaying said output signal in at least one of a graphical format and a numerical format.

26. A computer program product tangibly stored on a computer-readable medium for facilitating non-invasive monitoring of intracranial pressure (ICP) variations in an intracranial compartment of a patient, comprising:
computer code for receiving at least one data signal representative of optical radiation that has migrated transcranially outward from the intracranial compartment after having been transcranially introduced thereinto; and
computer code for processing said at least one data signal to generate therefrom an output signal representative of said ICP variations, wherein said processing said at least one data signal comprises (i) extracting therefrom at least one component signal that varies in time according to one of an intrinsic physiological oscillation in the patient and an externally driven oscillation in the patient, and (ii) computing said output signal based at least in part on an amplitude characteristic of each of said extracted component signals.

27. The computer program product of claim 26, wherein said at least one extracted component signal consists of a single component signal corresponding to a single intrinsic physiological oscillation in the patient or a single externally driven oscillation in the patient, and wherein said computing said output signal comprises:
detecting an amplitude characteristic of said single component signal;
assigning a decreasing value for said output signal as said amplitude characteristic increases; and
assigning an increasing value for said output signal as said amplitude characteristic decreases.

28. The computer program product of claim 26, wherein said single component signal varies in time according to one of an intrinsic respiratory oscillation, a cardiac oscillation, and a ventilated respiratory oscillation.

29. The computer program product of claim 25, wherein said single component signal varies in time according to an oscillation frequency of an external mechanical vibrator disposed in mechanical communication with the head of the patient.

30. A method for monitoring an intracranial pressure (ICP) of a patient, comprising:
monitoring an absolute ICP level of the patient using an invasive ICP monitoring device, the invasive monitoring device requiring the placement of an invasive instrument through a hole in the patient's skull;
bringing optical radiation-based non-invasive ICP monitoring device into optical communication with the patient's head while the invasive instrument of the invasive ICP monitoring device is still in the patient's skull;
using absolute ICP levels determined by the invasive ICP monitoring device for calibrating the non-invasive ICP monitoring device;
removing the invasive monitoring device from the patient including removing the invasive instrument from the hole in the patient's skull; and
subsequent to said removing, continuing to monitor the ICP of the patient using the non-invasive ICP monitoring device as calibrated by the invasive ICP monitoring device.

31. The method of claim 30, wherein said non-invasive ICP monitoring device is configured and adapted to use optical radiation to transcranially detect variations in the magnitudes of periodic intracranial matter oscillations that are intrinsically induced by patient physiology and/or extrinsically induced by external devices, the magnitude variations being indicative of intracranial matter compliance variations brought about by ICP changes.

32. A method for non-invasive detection of intracranial pressure (ICP) variations in an intracranial compartment of a patient, comprising:
applying a plurality of discrete mechanical impulses to the head of the patient at a respective plurality of discrete points in time;
during each of a plurality of time intervals immediately subsequent to each respective discrete point in time, applying optical radiation to the patient that propagates transcranially into the intracranial compartment, and detecting optical radiation that has migrated transcranially outward from the intracranial compartment; and
processing a plurality of time signals respectively representative of the optical radiation detected during said plurality of time intervals to generate an output signal representative of said ICP variations.

33. The method of claim 32, wherein said processing the plurality of time signals comprises:
for each said time signal, computing at least one transient characteristic thereof induced by the mechanical impulse associated therewith; and
on an impulse over impulse basis, assigning a decreasing value for said output signal when said at least one computed transient characteristic changes toward values indicative of greater intracranial matter compliance, and assigning an increasing value for said output signal when said at least one computed transient characteristic changes toward values indicative of lesser intracranial matter compliance.
